(19) United States
(12) Patent Application Publication
(10) Pub. No.: US 2019/0328536 A1
(43) Pub. Date: Oct. 31, 2019

(54) Reversed Joint Prosthetic With Cup Having a Centered and Aligned Post

(71) Applicant: FX Shoulder USA Inc., Farmers Branch, TX (US)

(72) Inventors: Baptiste Martin, Dallas, TX (US); Howard Walker Harris, Bedford, TX (US); Michael Patrick Bradley, Wakefield, RI (US); Umasuthan Srikumaran, Ellicott City, MD (US); Todd Christopher Moen, Dallas, TX (US); William Eric Carlson, Stuart, FL (US); Wayne Burkhead, JR., Dallas, TX (US)

(21) Appl. No.: 16/397,751

(22) Filed: Apr. 29, 2019

Related U.S. Application Data
(60) Provisional application No. 62/663,748, filed on Apr. 27, 2018.

(51) Int. Cl. A61F 2/40

(52) U.S. Cl. CPC A61F 2/4014; A61F 2002/30654

(57) ABSTRACT

A prosthetic device configured to function as a joint has a first joint component configured to be fixed to a first bone and a second joint component configured to engage the first joint component. The second joint component has an anchoring base configured to be fixed to a second bone, and a cup configured to be connected to the anchoring base. The cup has a cylindrical wall defining an outer surface of the cup, a cavity formed by a spherical recess inside of a circular edge at a top of the cylindrical wall, and an attachment post extending away from the cavity and configured to connect to the anchoring base. The attachment post is aligned with a center of a circle defined by the circular edge and an axis of the attachment post is parallel to the cylindrical wall.

### CROSS-REFERENCE TO RELATED APPLICATIONS

This application claims the benefit of U.S. Provisional Application Ser. No. 62/663,748 filed Apr. 27, 2018, which is incorporated herein by reference in its entirety.

### TECHNICAL FIELD

The present invention relates generally to joint prosthetics, and, more particularly, to a reversed joint prosthetic which includes a cup having a centered and aligned post.

### BACKGROUND

There are many situations in which a patient may benefit from a medical procedure in which a prosthetic device is inserted into a joint to reform, reshape, or otherwise improve the functioning of the joint. Common prosthetic joint procedures include shoulder or hip replacement. In many cases, the prosthetic is formed as an anatomical replacement. That is, the implanted device is formed to mimic the functioning of the natural joint. For example, the end of the humerus may be reformed with a dome-shaped prosthesis to match the natural shape of the bone while the glenoid cavity of the scapula is replaced or resurfaced with a glenoid implant.

While the anatomical joint prosthetic is applicable in many situations, a “reversed” procedure has emerged as an alternative for joint replacement. In the reversed procedure, the “ball” and “socket” of the natural joint are switched. In particular, a socket-like humeral cup may be secured to the end of the humerus and a ball-like glenosphere attached to the scapula. The humeral cup is configured to articulate with respect to the surface of the glenosphere, thereby allowing the shoulder joint to function. The reversed procedure and design may be more practical and effective than the anatomical version for many patients, especially those with certain muscle injuries because the reversed version relies on different muscles to move the arm.

The variation of bone and joint shape, structure, and condition across patients has led to the creation of interchangeable prosthetic parts of different sizes and shapes which allow a practitioner to choose a combination which is best suited for an individual. A standardized implant may be used in combination with each of these different parts in order to simplify the procedure. For example, different sizes and configurations of humeral cups may each be configured to fit and mate with the same anchoring base which is implanted into the humerus. In this way, each procedure may involve the same or similar steps for attaching the anchoring base to the bone. The selected humeral cup is then attached to the anchoring base.

The arrangement and connection between the anchoring base and the joint component should be carefully considered to achieve a reliable, long-lasting connection which avoids inducing stresses or forces which may loosen or move the anchoring base or cause a misalignment of the joint. The present disclosure includes embodiments of a prosthetic device and associated procedure which is tailored to provide a robust and reliable connection between prosthetic components.

### SUMMARY

In one aspect, an embodiment of the present disclosure is directed to a prosthetic device configured to function as a joint. The prosthetic device includes a first joint component configured to be fixed to a first bone and a second joint component configured to engage the first joint component. The second joint component includes an anchoring base configured to be fixed to a second bone, and a cup configured to be connected to the anchoring base. The cup includes a cylindrical wall defining an outer surface of the cup, a cavity formed by a spherical recess inside of a circular edge at a top of the cylindrical wall, and an attachment post extending away from the cavity and configured to connect to the anchoring base. The attachment post is aligned with a center of a circle defined by the circular edge and an axis of the attachment post is parallel to the cylindrical wall.

In another aspect, an embodiment of the present disclosure is directed to a humeral cup for a prosthetic shoulder joint. The cup includes a cylindrical wall defining an outer surface of the cup, a cavity formed by a spherical recess inside of a circular edge at a top of the cylindrical wall, and an attachment post extending away from the cavity and configured to connect to the anchoring base. The attachment post is aligned with a center of a circle defined by the circular edge and an axis of the attachment post is parallel to the cylindrical wall.

Additional features and advantages of the invention will be made apparent from the following detailed description of illustrative embodiments that proceeds with reference to the accompanying drawings.

### BRIEF DESCRIPTION OF THE DRAWINGS

The foregoing and other aspects of the present invention are best understood from the following detailed description when read in connection with the accompanying drawings. For the purpose of illustrating the invention, there are shown in the drawings embodiments that are presently preferred, it being understood, however, that the invention is not limited to the specific instrumentalities disclosed. Included in the drawings are the following Figures:

FIG. 1 is a side view of a prosthetic device, according to a disclosed embodiment, including associated bones;

FIG. 2 is a side view of the prosthetic device of FIG. 1, without the associated bones;

FIG. 3A is a perspective view of an exemplary anchoring base of the prosthetic device, according to an embodiment;

FIG. 3B is a side view of the anchoring base of FIG. 3A;

FIG. 3C is a top view of the anchoring base of FIG. 3A;

FIG. 4A is a perspective view of an exemplary cup of the prosthetic device, according to an embodiment;

FIG. 4B is a side view of the cup of FIG. 4A;

FIG. 4C is a top view of the cup of FIG. 4A;

FIG. 4D is a bottom view of the cup of FIG. 4A; and

FIG. 4E is a side cross-sectional view of the cup of FIG. 4A.

### DETAILED DESCRIPTION

A prosthetic device consistent with the disclosed embodiments includes a cup configured to mate with an implantable anchoring base. The anchoring base includes an implanted side which is inserted into a bone and a connection side which interfaces and connects to the cup. The anchoring base includes a cavity which is configured to receive a mating element on a bottom side of the cup, such as a post. A functional side of the cup includes a spherical recess which forms a joint cavity. Both a location and an axis of the attachment post is aligned at a center of the cavity, such that forces applied to the cup are evenly distributed to the anchoring base.

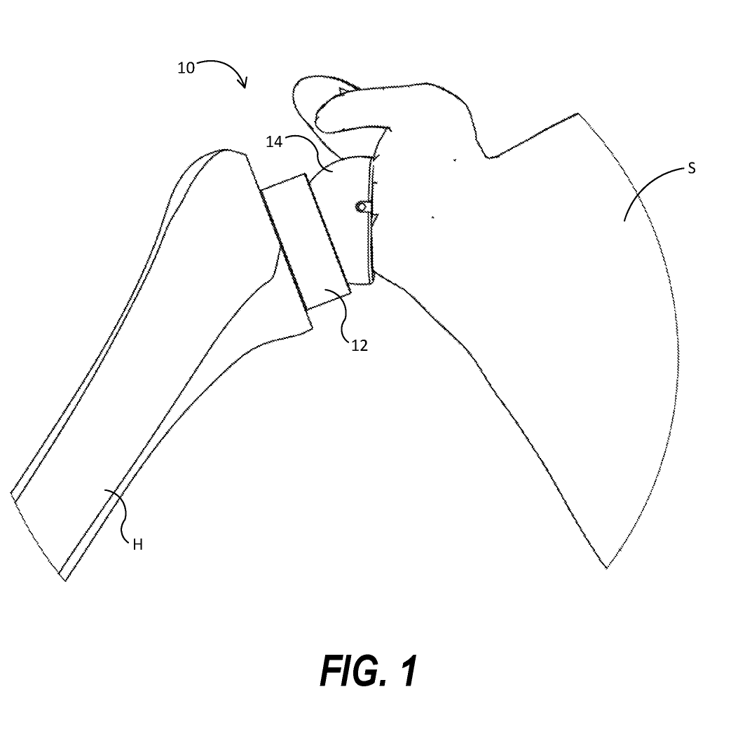

FIG. 1 is a side view of a prosthetic device 10 consistent with disclosed embodiments. The device 10 is a shoulder joint replacement positioned between the humerus H and the scapula S and includes several components which function a joint. In one embodiment, the device 10 includes a cup 12 and a glenosphere 14. The cup 12 is fixedly attached to the humerus H and the glenosphere 14 is fixedly attached to the scapula S. The cup 12 serves as the “socket” and the glenosphere 14 serves as the “ball” of the shoulder ball-and-socket joint. In this way, the device 100 is a reversed joint prosthetic, as a natural shoulder joint includes the opposite configuration. A reversed joint prosthetic, such as those commonly used in shoulder replacement procedures, allows a patient to rely on different muscles to move their arm. For example, if a patient has a weakened or damaged rotator cuff, a reversed joint prosthetic allows the patient to rely on the deltoid muscle to move and maintain the position of their arm.

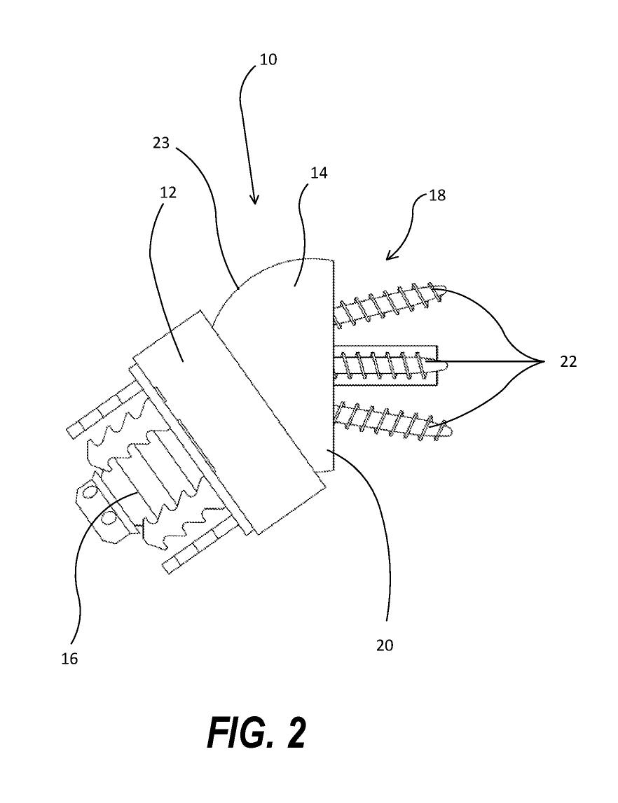

FIG. 2 is another side view of the prosthetic device 10 without the associated bones in view. An anchoring base 16 is connected to a bottom side of the cup 12. The anchoring base 16 is configured to be implanted into the humerus H in order to create a connection point for the cup 12. The anchoring base 16 may be a standardized component configured to mate with any number of different sizes and configurations of cups 12. In some embodiments, the anchoring base 16 may an Easytech™ anchoring base sold by FX Solutions.

The glenosphere 14 includes a securing member 18 configured to fix the glenosphere 14 to the scapula S. In an exemplary embodiment, the securing member 18 includes a base 20 and a plurality of screws 22 which secure the base 20 and the glenosphere 14 to the bone. The glenosphere 14 includes an surface 23 having a generally concave shape.

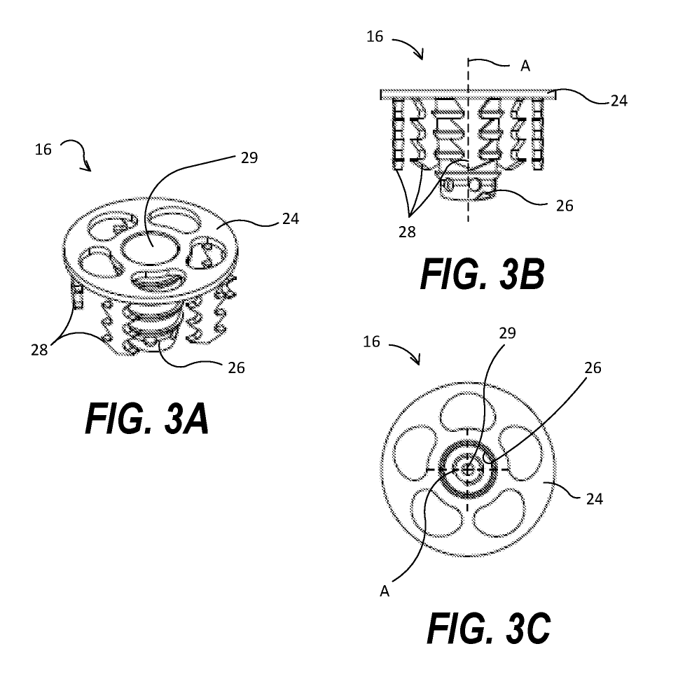

FIGS. 3A, 3B, and 3C further illustrate the anchoring base 16. The anchoring base 16 preferably includes a base surface 24, a central retaining peg 26, and a plurality of peripheral retaining spikes 28. The central retaining peg 26 and the peripheral retaining spikes 28 are implanted into the humerus H such that the base surface 24 is positioned at a surface of the bone. The central retaining peg 26 is hollow and includes an opening 29 at the base surface 24. The opening 29 is located at a center of the circular base surface 24. The retaining peg 26 extends perpendicular to the base surface 24 and is centered with respect to base surface 24. In other words, an axis A of the central retaining peg 26 passes through a center of the circular base surface 24. Moreover, the axis A is centered with respect to the center of the circle defined by the circular edge 40 of the cup 12 when the cup 12 is attached to the anchoring base 16.

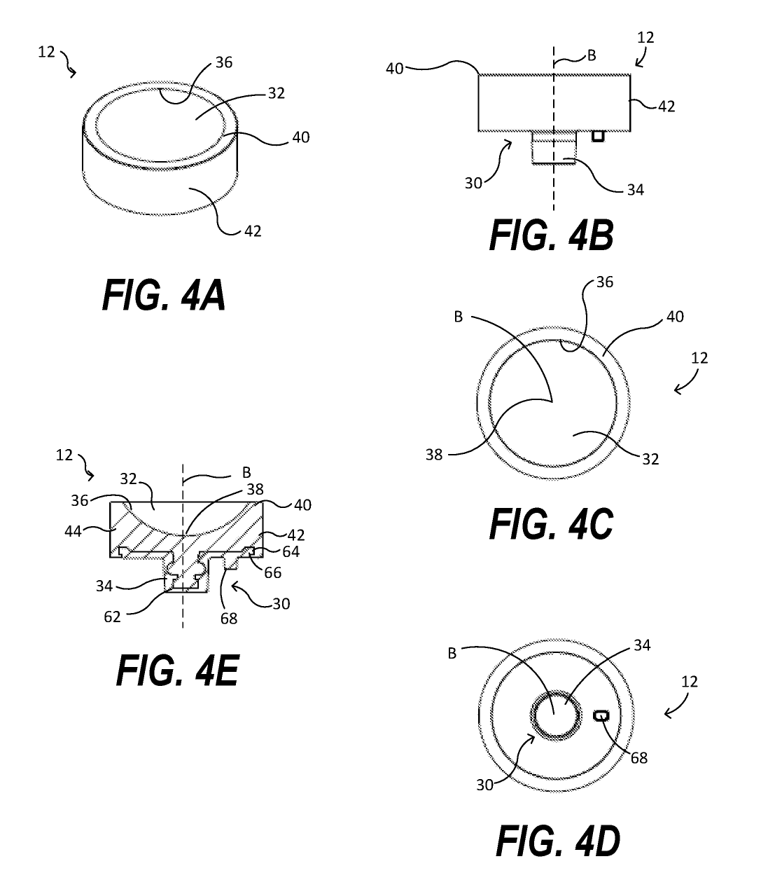

FIGS. 4A, 4B, 4C, 4D, and 4E further illustrate the cup 12. The cup 12 includes a mating element 30 on a bottom side of the cup 12 and a cavity 32 on a functional side of the cup 12. The mating element 30 may include an attachment post 34 which extends away from the bottom side of the cup 12. The attachment post 34 is configured to be received in the central retaining peg 26 in order to connect the cup 12 to the anchoring base 16, such as by an interference fit. In some aspects, the combination of the anchoring base 16 and cup 12 may be considered a stemless implant.

FIG. 4E is a cross-sectional side view of the cup 12. The cavity 32 is spherical such that the cavity 32 forms an articulating surface 36 configured to contact the concave surface 23 of the glenosphere 14 and form the interface of the shoulder joint created by the prosthetic device 10. The cavity 32 is formed by a spherical recess in which a low point 38 of the cavity 32 is centered with respect to a circular outer edge 40 of the cup 12. In other words, the bottom of the cavity 32 (i.e., the low point 38) is located at the center of a circle defined by the circular outer edge 40.

The spherical recess of the cavity 32 generally matches the shape of the glenosphere 14, preferably with some clearance to allow movement. A smaller clearance will create a more stable joint, while a larger clearance will provide greater mobility. The relative dimensions of the cavity 32 and the glenosphere 14 and the clearance therebetween can be customized depending on the individual factors of the patient. As shown, the convex shape of the surface 36 of cup 12 receives and cooperates with the concave shape of the surface 23 of glenosphere 14 to allow movement of the cup 12 relative to the glenosphere 14, and hence the humerus H with respect to the scapula S.

The attachment post 34 is also centered with respect to the cavity 32. In particular, the attachment post 34 extends in a perpendicular direction away from a top surface of the cup 12. In other words, the attachment post 34 extends parallel to a height direction of a cylindrical outer surface 42 of the cup 12 formed by a cylindrical wall. In addition, the attachment post 34 is centered with respect to the cavity 32. For example, an axis B of the attachment post 34 aligns with and passes through the low point 38 of the cavity 32 and the center of the circle formed by the circular outer edge 40 of the cup 12. In addition, the axis B of the attachment post 34 and the axis A of the central retaining peg 26 are collinear when the cup 12 is attached to the anchoring base 16. In alternative embodiments, the axis B of the attachment post 34 may be offset with respect to the cylindrical wall forming the outer surface 42 of the cup 12, while maintaining a centered relationship to the cavity 32.

In an exemplary embodiment, the cup 12 includes a body 44 formed of a thermocompressed plastic material and includes a shell 62 which is attached to the bottom side of the cup 12. The shell 62 may be formed of titanium and forms the attachment post 34. The plastic of the cup 12 may be overmolded onto the shell 62. The cup body 44 may further include a recessed groove 64 that extends around an edge 66 of the shell 62 (e.g., such that the plastic or other material of the cup body 43 covers the edge 66 of the shell 62). The shell 62 may also include an optional alignment element 68 that helps to align the cup 12 with respect to the anchoring base 16.

The cup 12 may be available in multiple sizes and/or other materials. For example, the cup 12 may be formed in two diameter sizes, Ø36 and Ø40 mm. Each size may be available in multiple versions depending on the status and condition of the patient. For example, the versions might include standard, mobility, and stability versions. The standard version may include a selected depth for the cavity 32, such as 7 mm or 8 mm. The depth may be measured from the top of the cavity 32 to the low point 38. The mobility version may include a slightly shallower cavity 32, such as a depth of 6mm. The shallower cavity 32 provides a greater range of motion to the patient. The stability version may include a slightly deeper cavity 32, such as a depth of 9 mm. The deeper cavity 32 allows the glenosphere 14 to sit deeper in the cup 12, providing greater stability to the joint. In some embodiments, the different versions relate to the location of the glenosphere within the cavity. For example, the center of the glenosphere may sit higher relative to a top edge of the cup in the mobility version, when compared to the standard version, while the center of the glenosphere may sit lower relative to the top edge of the cup in the stability version, when compared to the standard version.

Each version may be available in multiple heights. For example, the heights may include: +3 mm, +6 mm, +9 mm. Preferably, the cup 12 is compatible with all sizes of attachment posts. For example, a 10.4 mm attachment post may be used for attachment of the cup 12 to the anchoring base 16. It should be understood that alternative configurations of the cup 12 are possible without exceeding the scope of this disclosure.

An exemplary procedure for fixing the prosthetic device 10 in place (i.e., a joint replacement procedure) may include preparing the humerus H and implanting the anchoring base 16 (or other anchoring element) in a manner known in the art. The procedure may also include selection of the cup 12 from a variety of different sizes (e.g., standard version, mobility version, stability version, different cup heights and/or diameters), each of which may include the centered configured shown in FIGS. 4A-E.

The attachment post 34 is connected to the central retaining peg 26, such as through an interference fit which locks the cup 12 in place on the humerus H. The glenosphere 14 is fixed to the scapula S in a manner known in the art.

As a result of the procedure, the patient may move their arm, causing the cup 12 to articulate around the outer surface of the glenosphere 14. The centered configuration of the cup 12 and the anchoring base 16 provide advantages over prior designs. In particular, the interface between the cup 12 and the anchoring base 16 is symmetrical, resulting in a centering of any force transfer between the cup 12 and the anchoring base 16. Stress or torque received at the articulating surface 36 of the cavity 32 is thus applied evenly to the anchoring base regardless of the location of the stress or torque, due to the symmetrical and aligned configuration. Moreover, the perpendicular alignment of the axes of the central retaining peg 26 and the attachment post 34 with the base surface 24 and the cavity 32 help to avoid the development of uneven forces on the anchoring base 16. The centering of the cup 12 with respect to the anchoring base 16 also helps to alleviate tension on the patient's deltoid muscle by centering the joint with respect to the humerus. Moreover, the patient may experience less bleeding and bone loss as a result of the centered attachment post 34.

In use, at least some pressure that is generated by the prosthetic device 10 is in the form of compression into the cancellous bone of the humerus. The anchoring base 16 is configured such that it does not sink into the bone because the base surface 24 of it is planar and arranged to be supported on the prepared end of the humerus. The centering of the cup 12 on the anchoring base 16 makes it easier for the practitioner to correctly install the cup 12, as the angular orientation of the cup will not change the relative positioning of the cup 12 and the anchoring base 16, helping to avoid situations of instability. Moreover, while the retaining spikes 28 inhibit the anchoring base 16 from rotating, the centering of the cup 12 on the anchoring base 16 discourages the transfer of rotational forces to the central retaining peg 26 by.

The centering of the cup 12 and the anchoring base 16 and alignment of the axes helps to inhibit the application of a torque on the anchoring base 16 that could cause the anchoring base to rotate and/or loosen from its implant site on the humerus H, and/or the cup 12 to rotate and/or loosen with respect to the anchoring base 16. Moreover, even if the cup 12 rotates, the location of the cavity 32 does not change, due to the symmetrical alignment.

It should be understood that the disclosed configuration may be implemented in other devices, such as other prosthetic devices for other joints. In one embodiment, the disclosed embodiments may include a prosthetic hip joint.

The elements of the figures are not exclusive. Other embodiments may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention.

1. A prosthetic device configured to function as a joint, comprising:
a first joint component configured to be fixed to a first bone;
a second joint component configured to engage the first joint component, the second joint component comprising:
an anchoring base configured to be fixed to a second bone; and
a cup configured to be connected to the anchoring base, the cup comprising:
a cylindrical wall defining an outer surface of the cup;
a cavity formed by a spherical recess inside of a circular edge at a top of the cylindrical wall; and
an attachment post extending away from the cavity and configured to connect to the anchoring base,
wherein the attachment post is aligned with a center of a circle defined by the circular edge.

2. The prosthetic device of claim 1, wherein a low point of the cavity is located at the center of the circle defined by the circular edge.

3. The prosthetic device of claim 1, wherein the anchoring base comprises a central retaining peg which is configured to receive the attachment post.

4. The prosthetic device of claim 3, wherein the anchoring base further comprises a circular base surface, and wherein the central retaining peg is centered with respect the circular base surface.

5. The prosthetic device of claim 4, wherein the central retaining peg is hollow and the circular base surface includes an opening into the central retaining peg, the opening being located at a center of the circular base surface.

6. The prosthetic device of claim 4, wherein the central retaining peg extends in a perpendicular direction with respect to the circular base surface.

7. The prosthetic device of claim 4, wherein the central retaining peg defines an axis which is centered with respect to the center of the circle defined by the circular edge of the cup when the cup is attached to the anchoring base.

8. The prosthetic device of claim 7, wherein the axis of the attachment post and the axis of the central retaining peg are collinear when the cup is attached to the anchoring base.

9. The prosthetic device of claim 3, wherein the anchoring base further comprises a plurality of retaining spikes positioned around the central retaining peg.

10. The prosthetic device of claim 1, wherein the first joint component comprises a glenosphere.

11. The prosthetic device of claim 1, wherein the joint is a reversed anatomical joint.

12. The prosthetic device of claim 10, wherein the first bone is a scapula and the second bone is a humerus.

13. The prosthetic device of claim 1, wherein an axis of the attachment post is parallel to the cylindrical wall.

14. The prosthetic device of claim 1, wherein an axis of the attachment post is offset with respect to the cylindrical wall.

15. A humeral cup of a prosthetic joint, comprising:
a cylindrical wall defining an outer surface of the cup;
a cavity formed by a spherical recess inside of a circular edge at a top of the cylindrical wall; and
an attachment post extending away from the cavity and configured to connect to the anchoring base,
wherein the attachment post is aligned with a center of a circle defined by the circular edge, and
wherein an axis of the attachment post is parallel to the cylindrical wall.
